(19) United States
(12) Patent Application Publication
(10) Pub. No.: US 2023/0094392 A1
(43) Pub. Date: Mar. 30, 2023

(54) RESPIRATORY THERAPY DEVICE AND FAN UNIT

(71) Applicant: Loewenstein Medical Technology S.A., Luxembourg (LU)

(72) Inventor: Angela GERLACH, Hamburg (DE)

(21) Appl. No.: 17/935,733

(22) Filed: Sep. 27, 2022

(30) Foreign Application Priority Data
Sep. 28, 2021 (DE) 102021004890.3

(51) Int. Cl. A61M 16/00

(52) U.S. Cl. CPC A61M 16/0066; A61M 2205/42; A61M 2205/103

(57) ABSTRACT

A respiratory therapy device comprising at least one fan unit for generating a respiratory airflow for carrying out respiratory therapy. The fan unit comprises a housing and at least one fan wheel rotatably mounted in the housing. Respiratory air is transported through a channel formed inside the housing, which housing comprises at least one structural element, which reduces the sound emission.

### CROSS-REFERENCE TO RELATED APPLICATIONS

The present application claims priority under 35 U.S.C. § 119 of German Patent Application No. 102021004890.3, filed Sep. 28, 2021, the entire disclosure of which is expressly incorporated by reference herein.

### BACKGROUND OF THE INVENTION

### 1. Field of the Invention

The present invention relates to a respiratory therapy device and fan unit.

### 2. Discussion of Background Information

Ventilators and respiratory therapy devices for ventilation or respiratory assistance or for coughing assistance have a fan unit for generating a respiratory airflow to carry out respiratory therapy. At least one rotatably mounted fan wheel having a plurality of blade elements is typically arranged in the housing of the fan unit.

In order that the treatment or therapy is not perceived to be annoying, the operating noises of the fan unit are to be as low as possible. For efficient pressure buildup and targeted respiratory therapy, it is moreover decisive that high speeds are achieved and speed adjustments of the fan wheel can take place as quickly as possible and on short notice.

Flow noises occur due to vortex shedding and turbulent inflows and outflows inside the housing. These flow noises are composed of a broadband noise component and overlaid tones. The rotation sound is caused in particular by the impeller blades, when the fluid flows out of the blade channels on the housing tongue of the housing.

In view of the foregoing, it would be advantageous to have available a respiratory therapy device which can fulfill the above-mentioned requirements as advantageously as possible.

### SUMMARY OF THE INVENTION

The invention provides a respiratory therapy device which comprises at least one fan unit for generating a respiratory airflow for carrying out respiratory therapy, wherein the fan unit comprises a housing and at least one fan wheel rotatably mounted in the housing, wherein the respiratory air is transported through a channel formed inside the housing, and wherein the housing has at least one structural element which reduces the sound emission.

In some embodiments, the respiratory therapy device is characterized in that the housing comprises at least one inner side 16i, 17i and the channel comprises a lumen, wherein the lumen of the channel is enclosed by the inner side 16i and/or 17i.

In some embodiments, the respiratory therapy device is characterized in that the at least one structural element is arranged in the lumen of the channel.

In some embodiments, the respiratory therapy device is characterized in that the at least one structural element is arranged in and/or on the inner side 16i, 17i.

In some embodiments, the respiratory therapy device is characterized in that the channel comprises an inlet channel, at least one flow channel, and at least one outlet channel, which have a communication connection to one another, wherein the channel forms a flow path having a flow direction.

In some embodiments, the respiratory therapy device is characterized in that the flow direction extends from the inlet channel to the flow channel to the outlet channel.

In some embodiments, the respiratory therapy device is characterized in that the structural element is formed as at least one partition wall and/or at least one counterbore.

In some embodiments, the respiratory therapy device is characterized in that the partition wall is arranged at least in sections in the lumen of the flow channel and/or in the lumen of the outlet channel.

In some embodiments, the respiratory therapy device is characterized in that the partition wall divides the flow channel and/or the outlet channel at least in sections at least partially into at least two channels.

In some embodiments, the respiratory therapy device is characterized in that the partition wall is arranged in the channel in such a way that a second tongue is formed in the flow channel or in the outlet channel.

In some embodiments, the respiratory therapy device is characterized in that the at least one counterbore is arranged in the inner side 16i and/or 17i in the region of the flow channel and/or the outlet channel.

In some embodiments, the respiratory therapy device is characterized in that the counterbores are formed by a depression in the inner side 16i and/or 17i.

In some embodiments, the respiratory therapy device is characterized in that the counterbores have a depth of from about 0.5 mm to about 2.5 mm, preferably of from about 1 mm to about 2 mm, particularly preferably about 1.4 mm.

In some embodiments, the respiratory therapy device is characterized in that the counterbores are round and/or oval.

In some embodiments, the respiratory therapy device is characterized in that the counterbores have a diameter of from about 1 mm to about 7 mm, preferably of from about 2 mm to about 5 mm, particularly preferably of about 4 mm.

In some embodiments, the respiratory therapy device is characterized in that the inner sides 16i and/or 17i have from 1 to about 100 counterbores, preferably from 1 to about 50, particularly preferably from about 4 to about 20 counterbores.

The fan unit according to the invention is provided for a respiratory therapy device, preferably as was described above. The fan unit comprises a housing and at least one fan wheel rotatably mounted in the housing. The fan unit is preferably designed here as described above for the respiratory therapy device according to the invention.

### BRIEF DESCRIPTION OF THE DRAWINGS

Further advantages and features of the present invention will become clear from the description of the illustrative embodiments, which are explained below with reference to the accompanying drawings. In the drawings,

FIG. 1 shows a schematic illustration of a respiratory therapy device according to the invention in a perspective view;

FIG. 2 shows a schematic outside view of a housing according to the invention from the front;

FIG. 3 shows a schematic outside view of a housing according to the invention from the rear;

FIG. 4 shows a longitudinal section through a housing according to the invention of an exemplary embodiment with an inside view of the rear part, wherein FIG. 4A shows a view with inserted fan wheel and without partition wall and FIG. 4B shows a view without inserted fan wheel and with a partition wall according to the invention;

FIG. 5 shows a longitudinal section through the housing from FIG. 4 with an inside view of the front part;

FIG. 6 shows an enlarged detail from FIG. 4B;

FIG. 7 shows a schematic outside view of the housing from FIGS. 4-6 from the side with perspective into a pressure nozzle;

FIG. 8 shows a cross section through the housing from FIGS. 4-7;

FIG. 9 shows a longitudinal section through a housing according to the invention of a further exemplary embodiment with an inside view of the rear part with inserted fan wheel;

FIG. 10 shows a longitudinal section through the housing of the further exemplary embodiment with an inside view of the front part;

FIG. 11 shows a schematic outside view of the housing corresponding to the further exemplary embodiment from FIGS. 9-10 from the side with perspective into a pressure nozzle;

FIG. 12 shows a cross section through the housing of the further exemplary embodiment from FIGS. 9-11;

FIG. 13 shows a longitudinal section through a housing according to the invention of an alternative exemplary embodiment with an inside view of the rear part with inserted fan wheel;

FIG. 14 shows a cross section through the housing of the alternative exemplary embodiment from FIG. 13;

FIG. 15 schematically shows a cross section through the housing wall of an inner side of the front part or an inner side of the rear part of the alternative exemplary embodiment from FIGS. 13/14.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

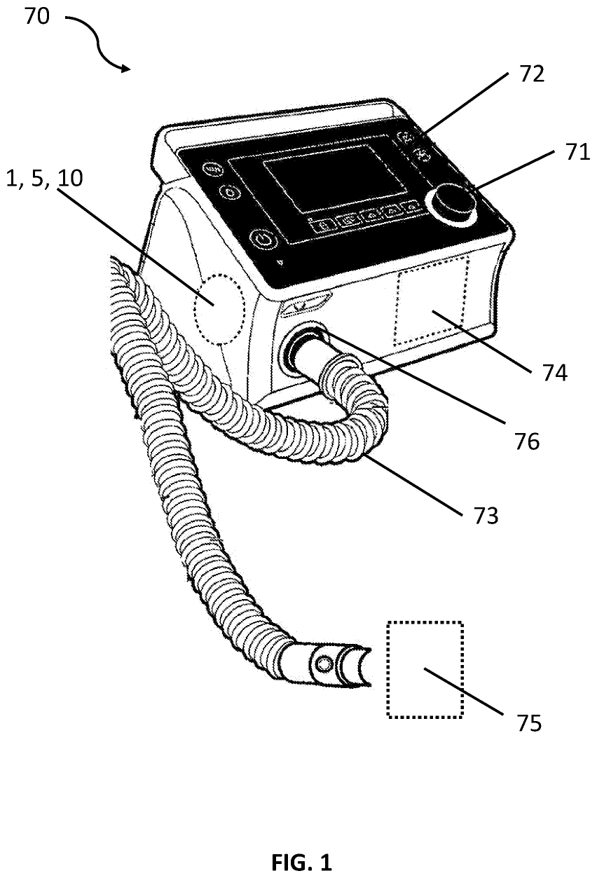

FIG. 1 shows a schematic illustration of a respiratory therapy device 70 according to the invention in a perspective view.

The respiratory therapy device 70 according to the invention is, for example, a ventilator for clinical or home applications, an emergency ventilator, a respiratory therapy device, or a coughing therapy device. The respiratory therapy device 70 is equipped with a fan unit 1 according to the invention (not visible here) housed in the device interior, using which a respiratory airflow for respiratory therapy is generated.

The fan unit 1 has a housing 10, in which a fan wheel 5 is arranged. The fan wheel 5 is arranged rotatably mounted in the housing 10. The fan wheel 5 comprises multiple blade elements 6. To rotate the fan wheel 5, the fan unit 1 has an electric drive which transmits rotational energy to the fan wheel 5. A pressure side and a negative pressure side arise at the fan wheel 5 due to the rotational movement, because of which the respiratory airflow occurs. Respiratory air in the meaning of the invention comprises any fluid, respiratory gas, and/or gas mixture which is suitable and can be used for ventilation, respiration, and/or respiratory therapy. In particular, the respiratory air can also be oxygen or can be air enriched with oxygen.

Fan units 1 which are used in respiratory therapy devices 70 are typically radial fans. In radial fans, a fluid, for example, respiratory air, enters the fan unit 1 in the axial direction and exits the fan unit 1 again perpendicularly to the axial direction. The fan unit 1 can also transport, in addition to respiratory air, any other gas mixture required for respiratory therapy.

The fan unit 1 is activated via a control unit 74 (not visible) arranged in the device interior. For example, the control unit 74 sets a specific speed of the fan wheel 5 or regulates the fan speed to a target value in dependence on the therapy specifications.

The respiratory therapy device 70 is equipped here with an operating unit 71 and with a display unit 72. A part of the operation takes place here, for example, via a touch-sensitive surface of the display unit 72.

The respiratory therapy device 70 has an interface 76 for coupling a hose system 73 for ventilation or coughing assistance. The respiratory airflow generated by means of the fan unit 1 is supplied via the hose system 73 to the patient (not shown). A patient interface 75 (not shown in greater detail here), for example a breathing mask, can be connected to the hose system 73.

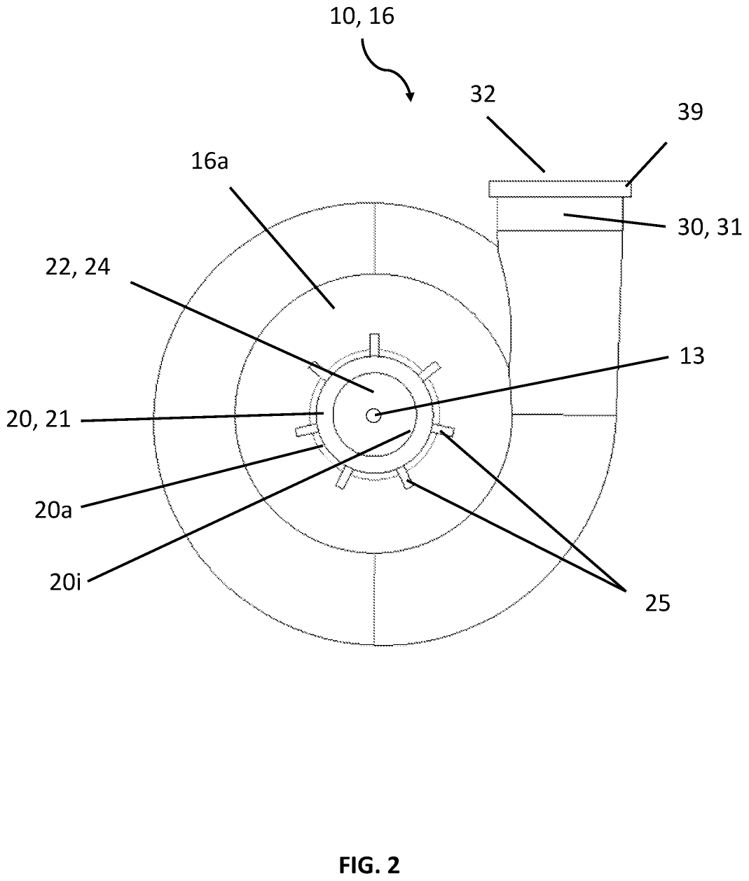

FIGS. 2-14 show various exemplary embodiments of the fan unit 1 according to the invention, wherein the housing 10 of the fan unit 1 is shown by way of example in different exemplary embodiments and in different views. The invention is not restricted to the illustrated exemplary embodiments.

The housing 10 of the fan unit 1 is generally spiral-shaped. The fan unit 1 comprises a suction nozzle 20 and a pressure nozzle 30. The fluid, for example respiratory air, can be aspirated via the suction nozzle 20. The fluid can be discharged via the pressure nozzle 30. The suction nozzle has a center axis 13. Respiratory air enters the fan unit 1 in the axial direction through the suction nozzle 20. The respiratory air exits the fan unit 1 again perpendicularly to the axial direction through the pressure nozzle 30.

The spiral-shaped housing 10 ensures that the respiratory air is collected in the housing 10 and conducted to the pressure nozzle 30, where it exits from the housing 10. Circular outlet flows, which result in losses, are prevented from occurring here. At the same time, a part of the speed energy of the respiratory air is converted into pressure energy by the spiral housing 10.

The spiral-shaped housing 10 has a housing wall 11 and comprises a front part 16 and a rear part 17. The front part 16 is by definition the element which comprises the suction nozzle 20. The rear part 17 is by definition the element which comprises an opening 40, through which a shaft of an electric motor can be connected to the fan wheel 5 (not shown).

Front part 16 and rear part 17 can be manufactured in one part or two parts. Front part 16 and rear part 17 are preferably manufactured in two parts. A two-part manufacturing facilitates the manufacturing and assembly. The fan unit 1 is in a usage state when front part 16 and rear part 17 are connected to one another and enclose a fan wheel 5 (not shown) in such a way that the fan wheel 5 is rotatably mounted.

Front part 16 and rear part 17 can be adhesively bonded, welded, screwed, clamped, pressed, or the like with one another, for example. Front part 16 and/or rear part 17 can also, for example, be connected to one another using an undercut. In this way, front part 16 and rear part 17 can be connected to one another. The connection can be reversible or irreversible. A connecting joint 18 can result due to the connection in the case of two-part manufacturing. The connecting joint 18 is preferably pronounced little enough that it does not cause impairment of the flow path.

The housing 10 can be produced from different materials. The selection of the material can have effects on the dimensional accuracy of the housing 10 and the roughness of the surfaces. The housing 10 can be manufactured, for example, from plastic and/or metal. For example, the housing 10 is manufactured from a plastic that can be injection molded. The housing 10 is preferably manufactured from polycarbonate and/or polyamide. The housing 10 is produced, for example, from polyamide PA12.

FIG. 2 shows a schematic outside view of a housing 10 according to the invention from the front. FIG. 2 shows the front part 16 of the housing 10. The front part 16 comprises an outer side 16a. The outer side 16a generally has an essentially smooth surface.

The front part 16 comprises the suction nozzle 20. The suction nozzle 20 has a suction nozzle wall 21 having a suction nozzle outer side 20a and a suction nozzle inner side 20i. The suction nozzle 20 forms an inlet channel 24 and comprises an inlet opening 22. The suction nozzle 20 can be formed as an oblong hollow body, in the lumen of which the inlet channel 24 extends. The suction nozzle inner side 20i comprises the lumen of the suction nozzle 20, in which the inlet channel 24 extends.

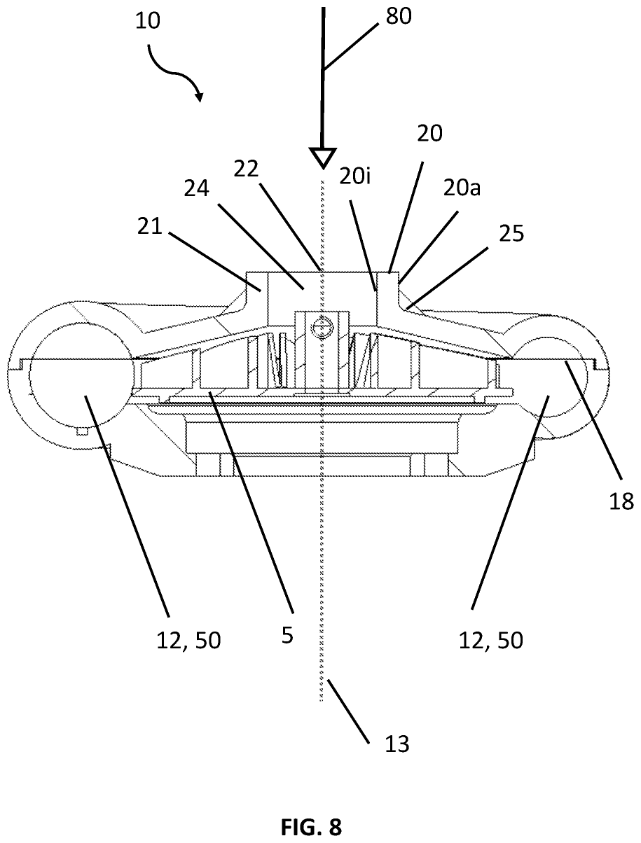

The diameter of the suction nozzle 20 can have any suitable geometry. In general, the diameter of the suction nozzle 20 is formed round, for example, circular. The suction nozzle 20 typically protrudes out of the outer contour of the housing 10 and exits out of the plane of the outer side 16a (see FIG. 8, for example).

A fluid, for example, a respiratory gas or gas mixture, can enter the housing 10 through the inlet channel 24 in the suction nozzle 20. Respiratory air can preferably enter the housing 10 via the inlet channel 24 and the suction nozzle 20. Ambient air can be aspirated via the suction nozzle 20, for example. In some embodiments, the suction nozzle 20 can be connected to a hose (not shown here), via which a defined gas and/or gas mixture can be introduced into the inlet channel 24.

To enable a hose connection to the suction nozzle 20, the suction nozzle 20 can comprise a suction nozzle flange 29. The suction nozzle flange 29 can be formed as an annular widening at the end of the suction nozzle (not shown).

The suction nozzle inner side 20i can be formed smooth, for example. In some embodiments, it is also conceivable that the suction nozzle inner side 20i is formed structured and has, for example, ribs, grooves, channels, or the like to optimize the flow properties of the fluid (not shown).

At least one, preferably multiple support ribs 25 can be arranged around the suction nozzle 20. The support ribs 25 can be arranged, for example, at least in sections on the suction nozzle outer side 20a. For example, the support ribs 25 can be arranged on the suction nozzle outer side 20a and on the outer side of the housing front part 16a and can connect these to one another in a supporting manner. The support ribs 25 are configured and formed to provide stability to the suction nozzle 20.

The housing 10 comprises the pressure nozzle 30. The pressure nozzle 30 has an outlet opening 32. The outlet opening 32 represents a pressure-side outflow opening out of the housing 10. A fluid, for example, a respiratory gas or gas mixture, can exit from the housing 10 through the pressure nozzle 30. Respiratory air can preferably exit through the pressure nozzle 30.

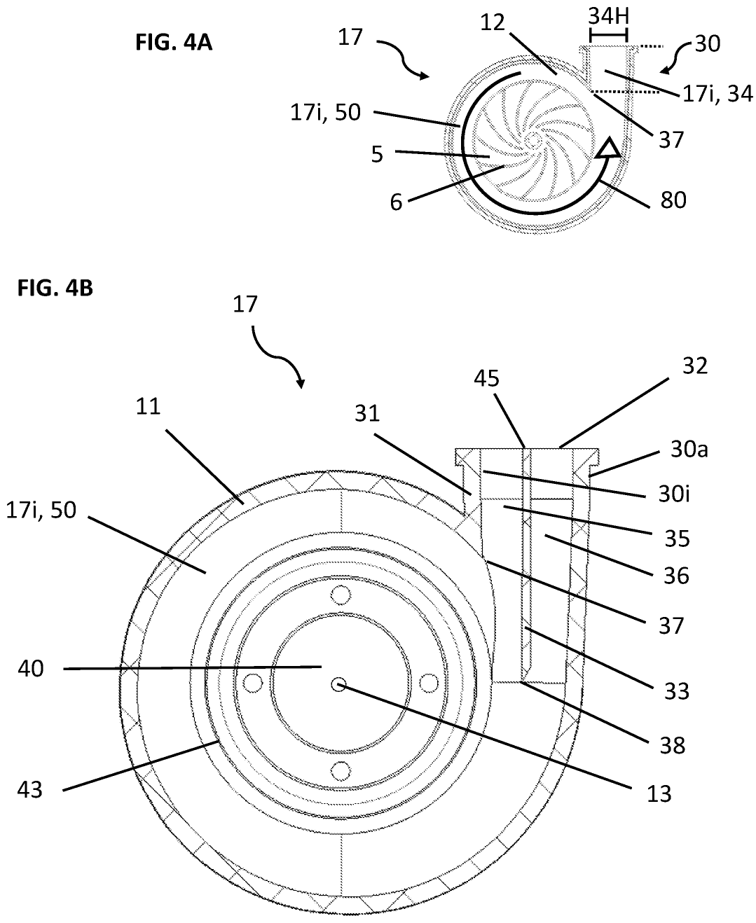

The pressure nozzle 30 has a pressure nozzle wall 31 having a pressure nozzle outer side 30a and a pressure nozzle inner side 30i (see FIG. 4, for example) and is generally formed at least partially as an oblong hollow body. The hollow body of the pressure nozzle 30 forms an outlet channel 34 (see FIG. 4, for example). The diameter of the pressure nozzle 30 can have any suitable geometry. In general, the diameter of the pressure nozzle 30 is formed round, for example, circular.

The pressure nozzle 30 can be connected to a hose or hose system 73 (not shown here), via which the respiratory air is discharged from the housing 10. The respiratory air can be supplied to the patient (not shown) via the hose system 73. To enable a hose connection to the pressure nozzle 30, the pressure nozzle 30 can comprise a pressure nozzle flange 39. The pressure nozzle flange 39 can be formed as an annular widening at the end of the pressure nozzle. The pressure nozzle flange 39 can be formed, for example, on the pressure nozzle wall 31.

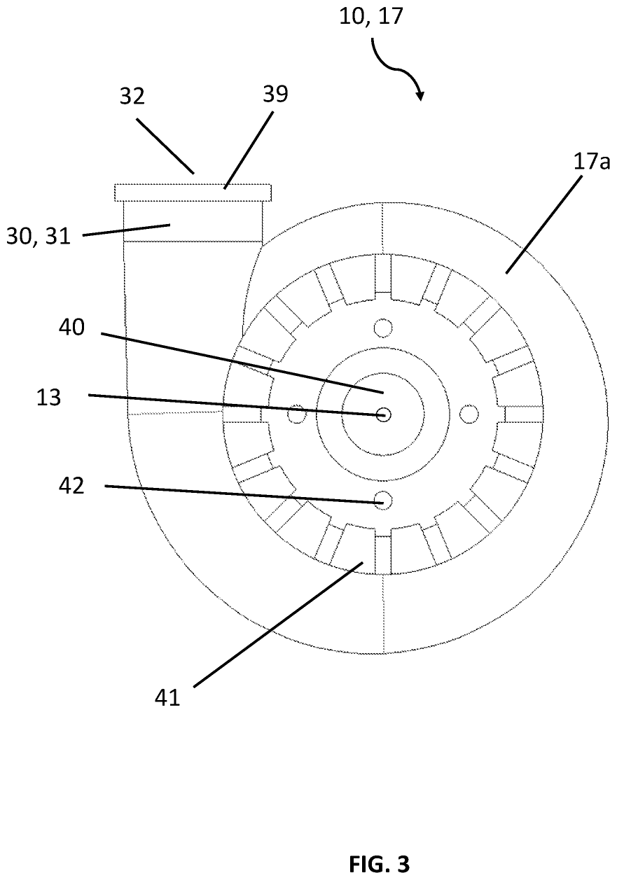

FIG. 3 shows a schematic outside view of a housing 10 according to the invention from the rear. FIG. 3 shows the rear part 17 of the housing 10. The rear part 17 comprises an outer side 17a. The outer side 17a generally has an essentially smooth surface.

The rear part 17 comprises at least one opening 40. A shaft of an electric motor can lead through the opening 40 and can be connected in the interior of the housing 10 to a fan wheel 5 in order to drive it (not shown). The opening 40 can be formed round, for example. In alternative embodiments, the opening 40 can also have any other suitable geometry.

Housing 10 and electric motor can be screwed together, for example. In one preferred embodiment, the housing 10 can be injection molded onto the electric motor.

The rear part 17 can have one or more bores 42. For example, the rear part 17 can have four bores 42. The bores 42 are preferably arranged radially around the opening 40. The housing 10 can be screwed together with the electric motor via the bores 42. One or more elements 41 can be arranged radially around the opening 40 for reinforcement.

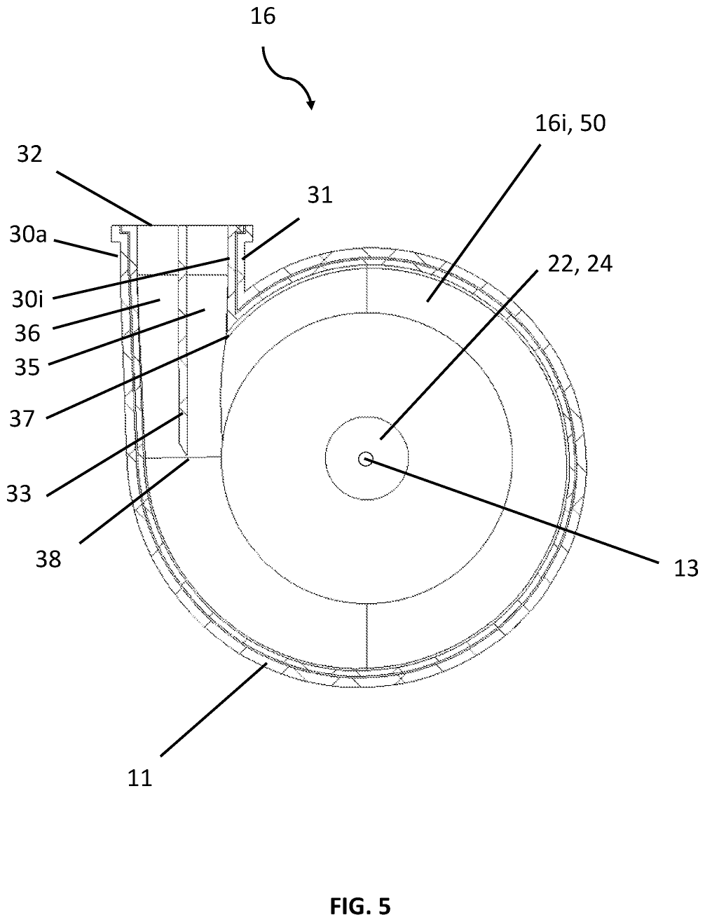

FIG. 4 shows a longitudinal section through a housing 10 according to the invention of an exemplary embodiment with an inside view of the rear part 17, wherein FIG. 4A shows a view with inserted fan wheel 5 and without partition wall 33 and FIG. 4B shows a view without inserted fan wheel 5 and with a partition wall 33 according to the invention. FIG. 5 shows a longitudinal section through the housing 10 from FIG. 4 with an inside view of the front part 16.

It is apparent from FIG. 4B that the rear part 17 can comprise a receptacle unit 43, which can accommodate the fan wheel 5. It is shown by way of example in FIG. 4A how the fan wheel 5 can be positioned to produce a usage state. The fan unit 1 is in a usage state when the fan wheel 5 is inserted into the receptacle unit 43 and front part 16 and rear part 17 are connected to one another. The fan wheel 5 is inserted here in such a way that the fan wheel 5 is rotatably mounted.

In general, the opening 40, through which the shaft of an electric motor that drives the fan wheel 5 leads, is arranged inside the receptacle unit 43. The center axis 13 can be located in the center of the opening 40.

The rear part 17 comprises an inner side 17i (FIG. 4). The surface of the inner side 17i can be formed smooth or at least partially structured. In general, the inner side 17i has an essentially smooth surface.

The front part 16 comprises an inner side 16i (FIG. 5). The surface of the inner side 16i can be formed smooth or at least partially structured. In general, the inner side 16i has an essentially smooth surface.

In a usage state, thus when front part 16 and rear part 17 are connected to one another, a channel 12 is configured and formed in the interior of the housing 10. The fluid, for example respiratory air, can flow through the channel 12. The channel 12 is formed as a hollow body. The channel 12 has a lumen. The lumen of the channel 12 is at least partially enclosed by the inner sides 16i and/or 17i.

The inner sides 16i, 17i can be curved to form the channel 12. For example, the inner sides 16i, 17i can be curved in such a way that the channel forms an essentially round channel 12. For example, the channel 12 has an essentially circular cross section (see FIG. 8, for example).

The pressure nozzle inner side 30i is a subsection of the inner sides 16i and 17i. The inner sides 16i and 17i, which enclose the lumen of the pressure nozzle 30, are also referred to herein as the pressure nozzle inner side 30i.

The channel 12 can comprise an inlet channel 24 (see FIG. 2), a flow channel 50, and an outlet channel 34 (see FIG. 4A). Inlet channel 24, flow channel 50, and outlet channel 34 are communicating channels. Inlet channel 24, flow channel 50, and outlet channel 34 are fluidically connected to one another. Inlet channel 24, flow channel 50, and outlet channel 34 form a flow path having a flow direction 80 (see FIG. 4A). For example, inlet channel 24, flow channel 50, and outlet channel 34 are pneumatically connected to one another. A fluid, for example respiratory air, can flow into the inlet channel 24 and can flow from there via the fan wheel 5 into the flow channel 50 and from the flow channel 50 into the outlet channel 34. The flow direction 80 generally extends from the inlet channel 24 via the flow channel 50 into the outlet channel 34.

In the case of two-part manufacturing of the fan unit 1, the flow channel 50 and/or the outlet channel 34 can be enclosed by the inner side of the front part 16i and by the inner side of the rear part 17i.

In a usage state, the inner sides 16i, 17i at least partially enclose the flow channel 50 at least in sections. In a usage state, the pressure nozzle inner side 30i at least partially encloses the outlet channel 34 at least in sections. The outlet channel 34 extends through the length of the pressure nozzle 30.

The pressure nozzle inner side 30i can be formed smooth, for example. In some embodiments, it is also conceivable that the pressure nozzle inner side 30i is formed structured and has, for example, ribs, grooves, channels, or the like to optimize the flow properties of the fluid (not shown).

The inner sides 16i, 17i and/or the pressure nozzle inner side 30i and/or the suction nozzle inner side 20i can have an essentially smooth surface in the region of the channel 12 in some embodiments. The surface can also have a material-related roughness. The nature of the surface can influence the pressure profile and the rotation sound.

The flow channel 50 is at least partially enclosed by the housing wall 11 in a usage state. The flow channel 50 extends circularly at least in sections. The flow channel 50 is connected to the inlet channel 24 and the outlet channel 34.

Respiratory air enters the fan unit 1 in the axial direction via the inlet opening 22 and the inlet channel 24. The inlet channel 24 extends linearly at least in sections, for example. From the inlet opening 22, the respiratory air moves via the inlet channel 24 into the fan wheel 5, which transports the respiratory air via a rotational movement into the flow channel 50. The respiratory air is transported in the radial direction via the substantially circular flow channel 50 and moves into the outlet channel 34. Respiratory air exits perpendicularly to the axial direction from the fan unit 1 via the outlet channel 34 and the outlet opening 32. A spiral-shaped flow direction 80 thus results.

A housing tongue 37 is formed where the housing wall 11 and the pressure nozzle wall 31 meet one another in the interior of the housing 10. By definition, the pressure nozzle 30 begins at the height of the housing tongue 37 (see FIG. 4A).

The respiratory air flows on the housing tongue 37, due to which a rotational sound can occur. Flow noises can also result inside the fan unit 1 due to vortex shedding and/or turbulent inflows and/or outflows. Furthermore, rotational sound results due to the blade elements 6 of the fan wheel 5 when they displace the respiratory air.

The housing tongue 37 forms a main source of the rotational sound. The position of the housing tongue 37 and/or the distance between fan wheel 5 and housing tongue 37 can have an influence here on the volume of the rotational sound.

According to the invention, the housing 10 has at least one structural element 33, 60, which reduces the sound emission.

The outlet channel 34 is at least partially enclosed by the pressure nozzle wall 31 in a usage state. The outlet channel 34 extends linearly at least in sections, for example. The outlet channel 34 has a height 34H. The outlet channel 34 is typically formed as a continuous channel having a height 34H (see FIG. 4A).

In one advantageous embodiment, at least the outlet channel 34 can be partitioned by at least one structural element 33 at least in sections into channels 35, 36 extending independently of one another (see FIG. 4B). The channels 35, 36 are described herein in more detail below. In alternative embodiments, the outlet channel 34 can also be partitioned by at least one structural element 33 into channels connected to one another.

In one embodiment according to the invention, the housing 10 of the fan unit 1 can comprise at least one structural element 33. The structural element can be formed in the form of a partition wall 33 (see FIG. 4B and thereafter).

The at least one partition wall 33 can be manufactured from the same material as the fan unit 1. In some embodiments, the partition wall 33 can also be manufactured from another suitable material. For example, the partition wall 33 can be manufactured from metal or plastic. The partition wall 33 can be formed on the front part 16 and/or on the rear part 17.

The partition wall 33 is arranged at least in sections in the channel 12. The partition wall 33 can be arranged at least in sections in the outlet channel 34. Alternatively or additionally, the partition wall 33 can be arranged in the flow channel 50.

The partition wall 33 can be formed in one part or two parts. In some embodiments, the partition wall 33 can be formed in two parts and can be formed on both the rear part 17 and also on the front part 16. In a usage state, the two-part partition wall 33 can be joined together to form a partition wall 33 completely partitioning the channel 12 at least partially. In some embodiments, the partition wall 33 can only incompletely divide the channel 12 at least in sections (not shown).

In one preferred embodiment, the partition wall 33 is formed in one part. In a usage state, thus when front part 16 and rear part 17 are connected to one another, the partition wall 33 formed in one part can completely divide the channel 12 at least in sections. The channel 12 can extend divided in two at least in sections by the partition wall 33.

The partition wall 33 can be in one part and can be formed on the rear part 17, for example. In this case, the front part 16 can have a receptacle joint 44 (not shown), in which the partition wall 33 can be inserted. Alternatively, a one-part partition wall 33 can also be formed on the front part 16. The rear part 17 can then have a receptacle joint 44, in which the partition wall 33 can be inserted (not shown).

The partition wall 33 can extend at least in sections through the channel 12 in the flow direction 80. The partition wall 33 has a beginning 38 and an end 45. The beginning 38 of the partition wall 33 is farther to the front with respect to the flow direction and the end 45 of the partition wall 33 is farther to the rear with respect to the flow direction 80.

In some embodiments, the end 45 can be arranged inside the housing (not shown). In the exemplary embodiments shown here, the end 45 can be arranged directly at the height of the outlet opening 32 (see, for example, FIG. 4B).

The beginning 38 lies inside the housing. The beginning 38 of the partition wall 33 is arranged in the channel 12. A second tongue 38 is introduced into the housing 10 by the beginning of the partition wall 33. The second tongue 38 is arranged inside the housing. The second tongue 38 results at the beginning of the partition wall 33. The beginning of the partition wall 33 and thus the second tongue 38 can be formed straight, rounded, and/or beveled.

The amplitude of the rotational sound can be reduced by the second tongue 38. The rotational sound can be allocated onto multiple frequencies by the second tongue 38. The sound subjectively perceived by the user/patient can thus be improved, since the tonality of the rotational sound becomes less. The pressure curve, in contrast, is not influenced by introducing a second tongue 38.

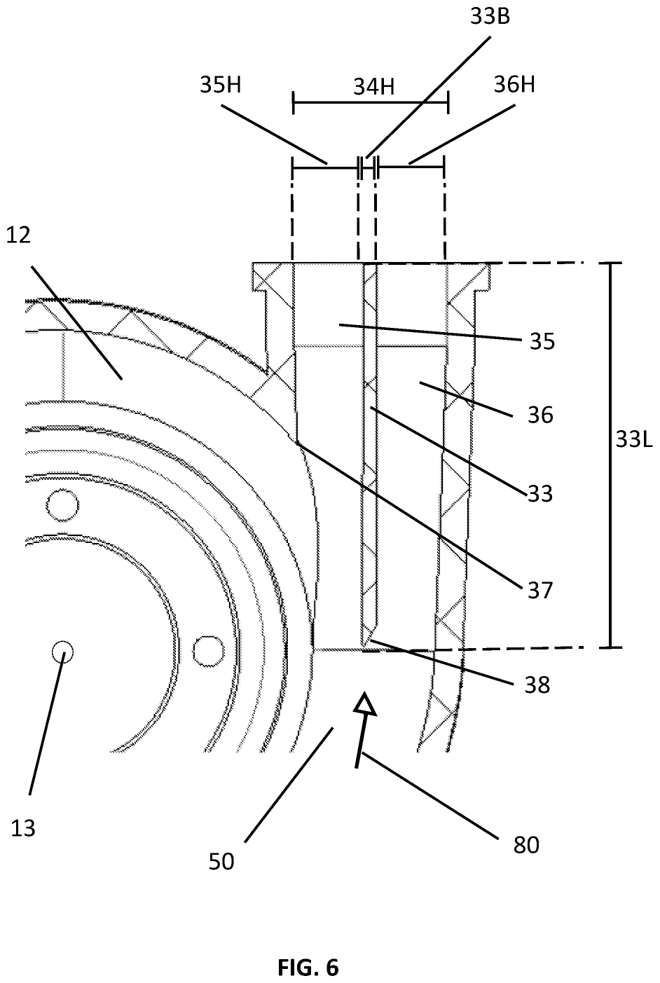

FIG. 6 shows an enlarged detail from FIG. 4B. The partition wall 33 can divide the channel 12 at least in sections along the flow direction 80. The partition wall 33 has a length 33L. The length 33L can be formed in different lengths. Depending on the length 33L, the partition wall 33 can divide the flow channel 50 and/or the outlet channel 34 at least in sections along the flow direction 80.

The lengths of the partition wall 33L can be selected such that from about 5% to about 100% of the outlet channel 34 is divided by the partition wall 33. Preferably, from about 20% to about 100% of the outlet channel 34 is divided by the partition wall 33, particularly preferably from about 50% to about 100% of the outlet channel 34 is divided by the partition wall 33.

In one specific exemplary embodiment, the length of the partition wall 33L is configured and formed in such a way that the partition wall 33 extends completely through the length of the outlet channel 34. 100% of the outlet channel 34 is divided by the partition wall 33 (see FIG. 6). The partition wall 33 can alternatively or additionally also extend at least partially through the flow channel 50.

The length of the partition wall 33L can be selected such that from 0% to about 90% of the flow channel 50 is divided by the partition wall 33. Preferably, from about 5% to about 80% of the flow channel 50 is divided by the partition wall 33.

The partition wall 33 can extend in the specific exemplary embodiment shown in FIG. 6 up to the height of the center axis 13. The partition wall 33 then extends through the outlet channel 34 and at least partially through the flow channel 50. In the specific exemplary embodiment shown in FIG. 6, 100% of the outlet channel 34 and additionally approximately 15% of the flow channel 50 are divided by the partition wall 33.

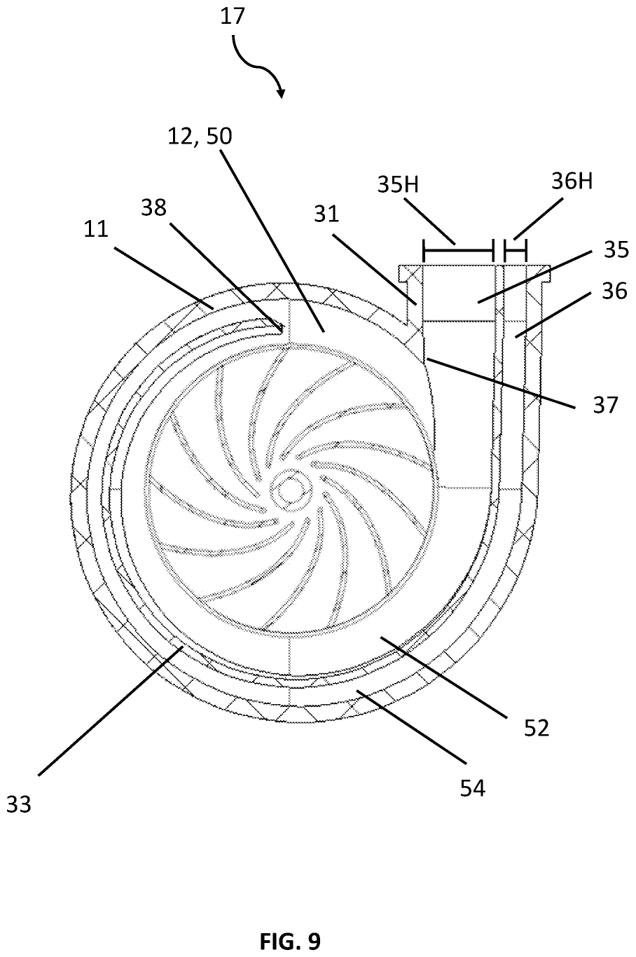

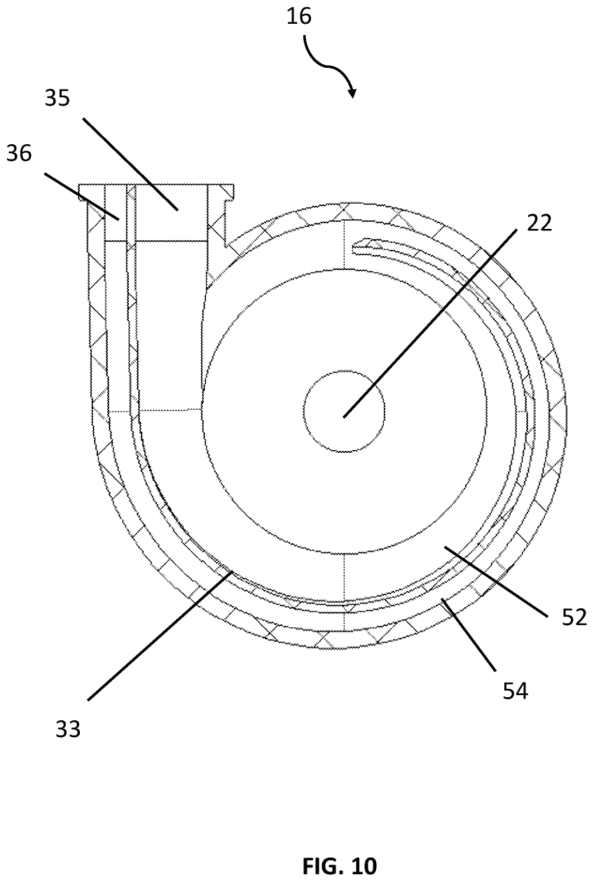

The partition wall 33 can extend in the specific exemplary embodiment shown in FIG. 6 up to the outlet opening 32. In some embodiments, the partition wall 33 can also be formed shorter and can end inside the outlet channel 34 and/or inside the flow channel 50 (not shown). In alternative exemplary embodiments, the partition wall 33 can also be formed longer (see FIGS. 9-10) or shorter (not shown).

The partition wall 33 has a width 33B. The width of the partition wall 33B can be as wide or wider than the housing wall 11. In one preferred embodiment, the width of the partition wall 33B is formed narrower than the housing wall 11. The width of the partition wall 33B is in a range of from about 0.1 mm to about 8 mm, preferably in a range of from about 0.5 mm to about 5 mm, particularly preferably in a range of from about 0.5 mm to about 2 mm. For example, the width of the partition wall 33B is 1 mm. The partition wall 33 is to be formed as thin as possible so as to keep the flow cross section of the channels as large as possible. The partition wall 33 is nonetheless to be formed thick enough that it has sufficient stability.

The partition wall 33 can partition the outlet channel 34 in such a way that an inner outlet channel 35 having a height 35H and an outer outlet channel 36 having a height 36H result. The respiratory air can be transported out of the housing via the outer outlet channel 36. The respiratory air can be transported out of the housing 10 and/or can move back into the flow channel 50 via the inner outlet channel 35. The arrangement of the partition wall 33 in the channel 12 can have an influence on the speed profile and/or on the pressure variations of the flow.

The partition wall 33 can be arranged centrally in the outlet channel 34. With a central arrangement, the outlet channel is divided in two and the height 35H of the inner outlet channel 34 corresponds to the height 36H of the outer outlet channel 36 (see FIG. 6).

In alternative embodiments, the height 35H of the inner outlet channel 35 can also be made less than the height 36H of the outer outlet channel 36. In some embodiments, the height 35H of the inner outlet channel 35 can also be made greater than the height 36H of the outer outlet channel 36 (not shown).

The location of the impeller 5 in relation to the partition wall 33 is caused by the radial arrangement of the partition wall inside the channel 12. If the partition wall 33 is arranged radially farther outward, the distance to the impeller 5 increases. If the partition wall 33 is arranged radially farther inward, the distance to the impeller 5 decreases. A greater distance of the partition wall 33 to the impeller 5 can be advantageous, since the speed profile can thus be smoothed and pressure variations at the tongue 37 can be reduced.

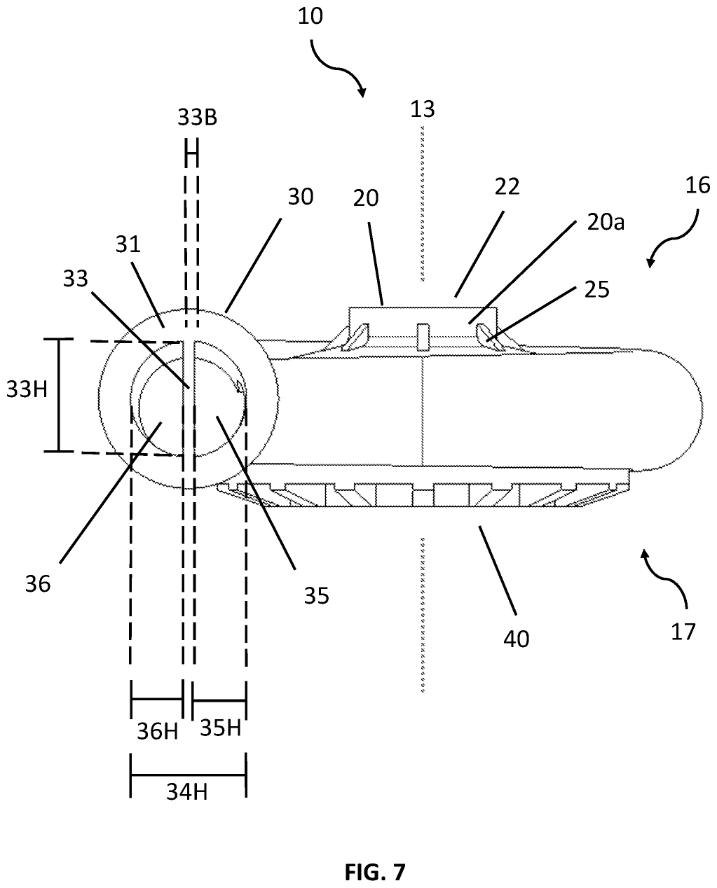

FIG. 7 shows a schematic outside view of the housing 10 from FIGS. 4-6 from the side with perspective into a pressure nozzle 30.

The partition wall 33 has a height 33H. The partition wall 33 can be formed continuously between front part 16 and rear part 17. In this case, the partition wall 33 is connected to the inner side of the front part 16i and to the inner side of the rear part 17i. The partition wall 33 can thus completely divide the channel 12 at least in sections. For example, the partition wall 33 completely partitions the outlet channel 34 into two channels 35, 36 extending independently of one another (FIG. 7).

In some embodiments, the height of the partition wall 33H can also be made less than the diameter of the outlet channel 34. The partition wall 33 then cannot be formed continuously and can only be connected to the inner side of the front part 16i or only to the inner side of the rear part 17i. The partition wall 33 can thus incompletely divide the channel 12 at least in sections (not shown).

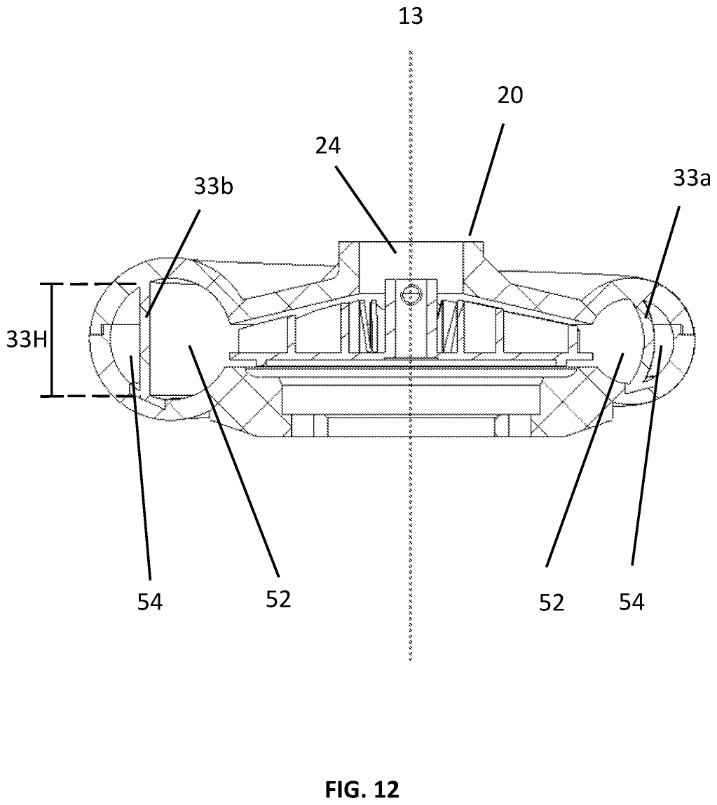

The partition wall 33 can be formed linearly along the height 33H (FIG. 7). In some embodiments, the partition wall 33 can also be formed convexly and/or concavely curved along the height 33H (see FIG. 12).

FIG. 8 shows a cross section through the housing 10 from FIGS. 4-7. It is apparent from FIG. 8 that the suction nozzle 20 protrudes out of the outer contour of the housing 10. The suction nozzle inner side 20i comprises the lumen of the suction nozzle 20, in which the inlet channel 24 extends. A fluid, for example, respiratory air, enters the housing 10 via the lumen of the suction nozzle 20. The flow direction 80 into the housing 10 is identified by an arrow. The flow direction 80 extends from the inlet channel 24 of the suction nozzle 20 into the impeller 5 and from the impeller 5 via the flow channel 50 into the outlet channel 34 (not shown here).

FIG. 9 shows a longitudinal section through a housing 10 according to the invention of a further exemplary embodiment with an inside view of the rear part 17 with inserted fan wheel and FIG. 10 shows a longitudinal section through the housing 10 of the further exemplary embodiment with an inside view of the front part 16.

In the specific exemplary embodiment according to FIGS. 9/10, the partition wall 33 is formed significantly longer than previously described. In this specific exemplary embodiment, the length of the partition wall 33L is configured and formed such that the partition wall 33 extends completely through the length of the outlet channel 34. The outlet channel 34 is divided by the partition wall 33 into an inner outlet channel 35 and an outer outlet channel 36. For example, 100% of the outlet channel 34 is divided by the partition wall 33.

In addition, the partition wall 33 can also be arranged at least in sections in the flow channel 50. The flow channel 50 can be divided by the partition wall 33 into an inner flow channel 52 and an outer flow channel 54.

In this exemplary embodiment, 100% of the outlet channel 34 and in addition approximately 80% of the flow channel 50 is divided by the partition wall 33. The spiral-shaped flow direction 80 can thus substantially extend in two parts. Because the partition wall 33 is formed sufficiently long that it is additionally arranged in the flow channel 50, the second tongue 38 is arranged well inside the housing 10. The second tongue 38 is located at the beginning of the flow direction 80. This offers the advantage that better flow guiding in the spiral is provided and/or fewer lateral movements of the fluid are possible.

In some embodiments, the partition wall 33 can also be arranged exclusively in the flow channel 50 and can at least partially divide the flow channel 50 (not shown).

The partition wall 33 can always be arranged centrally in the channel 12 in some embodiments (not shown). The arrangement of the partition wall 33 inside the channel 12 can change with the course of the flow direction 80 (see FIG. 9, for example). In the specific exemplary embodiment shown here, the partition wall 33 is arranged such that the outer flow channel 54 remains nearly uniform in its width, whereas the inner flow channel 52 successively increases in its width. The partition wall is accordingly not arranged centrally in the outlet channel, but rather displaced radially outward. The height of the outer outlet channel 36H can thus be formed smaller than the height of the inner outlet channel 35H.

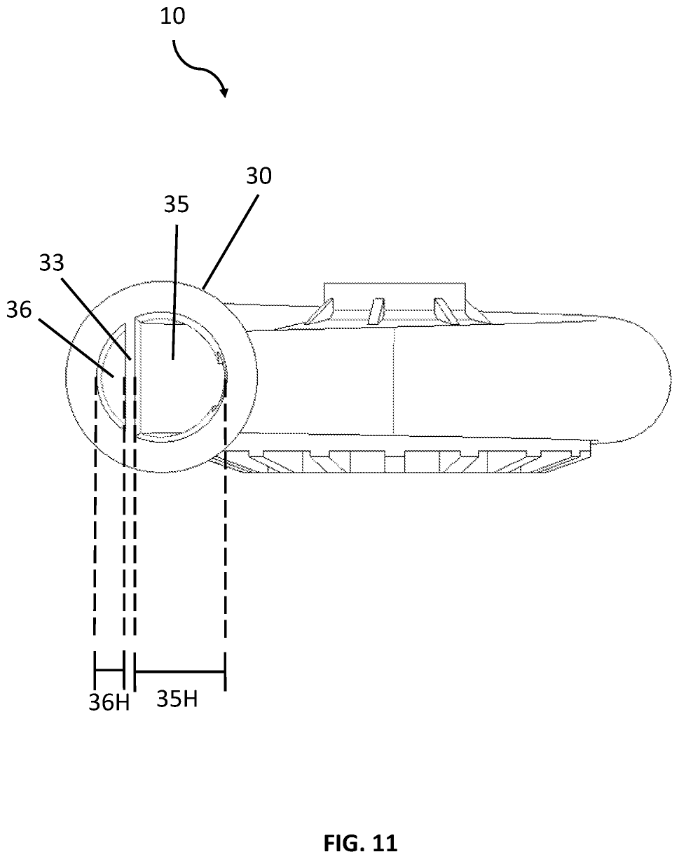

FIG. 11 shows a schematic outside view of the housing 10 corresponding to the further exemplary embodiment from FIGS. 9-10 from the side with perspective into a pressure nozzle 30. It is apparent from FIG. 11 that the partition wall 33 does not have to be arranged centrally in the pressure nozzle 30. The partition wall 33 is arranged radially outward in this exemplary embodiment. The height of the inner outlet channel 35H is therefore greater than the height of the outer outlet channel 36H. In alternative embodiments, the partition wall 33 can also be arranged centrally. The height of the inner outlet channel 35H can then be equal to the height of the outer outlet channel 36H (see FIG. 7). In some embodiments, the partition wall 33 can also be arranged radially inward. The height of the inner outlet channel 35H can then be less than the height of the outer outlet channel 36H (not shown). Moreover, it is apparent from FIG. 7 that the partition wall 33 is formed linearly in the pressure nozzle 30. In some embodiments, the partition wall 33 can alternatively or additionally also be formed convexly and/or concavely curved at least in sections (see FIG. 12).

FIG. 12 shows a cross section through the housing 10 of the further exemplary embodiment from FIGS. 9-11. It is apparent from FIG. 12 that the partition wall 33 is also arranged in the flow channel 50. Due to the arrangement of the partition wall 33 in the flow channel 50, it is partitioned into an inner flow channel 52 and an outer flow channel 54. In this specific exemplary embodiment, the partition wall 33 is not arranged centrally in the flow channel 50, but rather increasingly displaced radially outward. The inner flow channel 52 can therefore be formed larger than the outer flow channel 54. In some embodiments, the partition wall 33 can also be arranged in the flow channel 50 such that inner flow channel 52 and outer flow channel 54 are formed equal in size and/or such that the inner flow channel 52 is formed smaller than the outer flow channel 54 (not shown). The ratio of the size of the inner flow channel 52 and the outer flow channel 54 can remain uniform in the course of the flow direction 80 (not shown) or change in relation to one another (FIGS. 9-12).

It is additionally apparent from FIG. 12 that the partition wall 33 can be formed linear and/or curved along the height 33H. In this specific exemplary embodiment, the partition wall 33 is initially formed curved (33a) and changes its formation in the further flow progression in such a way that the partition wall 33 is formed linear (33b).

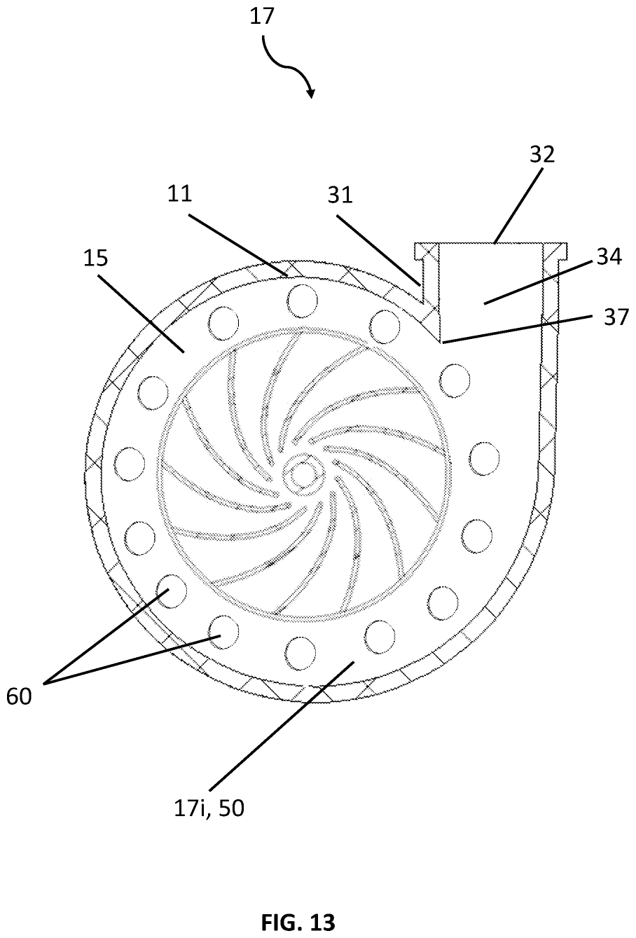

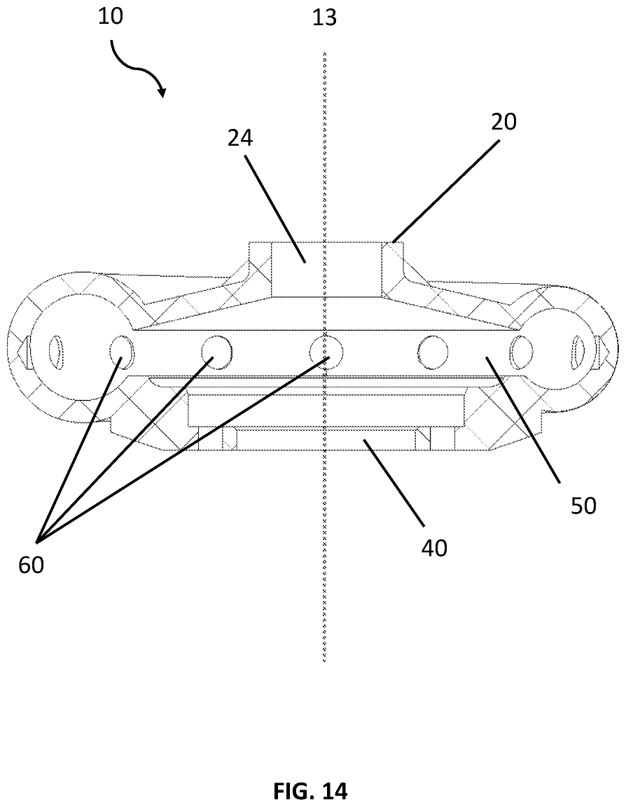

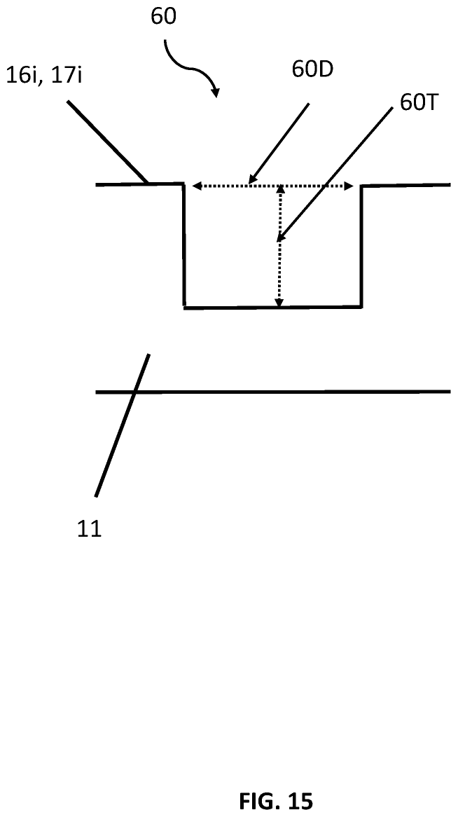

FIG. 13 shows a longitudinal section through a housing 10 according to the invention of an alternative exemplary embodiment with an inner view of the rear part 17 with inserted fan wheel 5. FIG. 14 shows a cross section through the housing 10 of the alternative exemplary embodiment from FIG. 13. FIG. 15 schematically shows a cross section through the housing wall 11 of an inner side of the front part 16i or an inner side of the rear part 17i of the alternative exemplary embodiment from FIGS. 13/14.

FIGS. 13-15 show a further embodiment according to the invention in which the housing 10 of the fan unit 1 can alternatively or additionally comprise at least one structural element 60. The structural element can be formed in the form of at least one counterbore 60. The one or more counterbores 60 can be arranged alternatively or additionally to the partition wall 33 in the housing 10.

The counterbores 60 can preferably be arranged on the flow channel 50 and/or on the outlet channel 34. The counterbores 60 can be arranged in or on the inner side 16i, 17i in the region of the flow channel 50 and/or the outlet channel 34. A structure can be introduced into the inner surface 16i, 17i by the counterbores 60, which can have a positive effect with respect to the rotational sound.

The counterbores 60 can be arranged on the inner side of the rear part 17i (FIG. 13) and/or on the inner side of the front part 16i (not shown). The counterbores 60 can be formed, for example, by a depression in the inner side of the rear part 17i and/or in the inner side of the front part 16i. The counterbores 60 have a depth 60T (see FIG. 15). The counterbores 60 are arranged at least on one of the inner sides of the housing 16i, 17i, preferably on both inner sides 16i, 17i. The counterbores 60 are configured and formed such that the otherwise essentially smooth surface of the inner sides 16i, 17i has sections which are provided in depressed form.

The counterbores 60 can be uniformly distributed. In some embodiments, the counterbores 60 can only be arranged at spots in specific regions or sections (not shown). The counterbores 60 can also be arranged randomly distributed over the inner sides 16i and/or 17i.

The counterbores 60 can have a depth 60T of from about 0.5 mm to about 2.5 mm. The counterbores 60 can preferably have a depth 60T of from about 1 mm to about 2 mm. For example, the counterbores 60 have a depth 60T of about 1.4 mm. The degree of the depression is selected such that the tonality is reduced. The degree of the depression is selected such that the overall sound pressure is reduced or remains the same. The degree of the depression is selected such that the characteristic curves can be improved. The counterbores 60 can all be formed equally deep or can vary in their depth 60T. The counterbores 60 can in some embodiments have deviating depths 60T at special regions of the inner sides 16i, 17i.

The depression of the counterbores 60 can extend linearly (see FIG. 15). In some embodiments, the depression of the counterbores 60 can taper and/or widen conically (not shown). The counterbores 60 can have a random shape in some embodiments.

The counterbores 60 can have any suitable geometry. In one preferred embodiment, the counterbores 60 can be formed round and/or oval. The counterbores 60 have a diameter 60D (see FIG. 15). The counterbores 60 can have a diameter 60D of from about 1 mm to about 7 mm. The counterbores 60 preferably have a diameter 60D of from about 2 mm to about 5 mm. For example, the counterbores 60 can have a diameter 60D of about 4 mm.

The inner side of the rear part 17i and/or the inner side of the front part 16i can have up to about 100 or more counterbores 60. The number of the counterbores 60 can be in a range of from 1 to about 100, preferably in a range of from 1 to about 50, particularly preferably in a range of from about 4 to about 20. For example, the inner side of the rear part 17i and/or the inner side of the front part 16i can each have 12 counterbores 60. The number of the counterbores 60 is selected such that the tonality decreases. The number of the counterbores 60 is selected such that the overall sound pressure is reduced or remains the same.

It is apparent from FIG. 14 that the counterbores 60 can in some embodiments alternatively or additionally also be arranged in the inner circumferential wall of the housing. The counterbores in the housing have the effect that the sound emission is significantly reduced.

To sum up, the present invention provides:
1. A respiratory therapy device, wherein the device comprises at least one fan unit for generating a respiratory airflow for carrying out respiratory therapy, the fan unit comprising a housing and at least one fan wheel rotatably mounted in the housing, and wherein the respiratory air is transported through a channel formed inside the housing, the housing comprising at least one structural element which reduces sound emission.
2. The respiratory therapy device of item 1, wherein the housing comprises at least one inner side and the channel comprises a lumen, the lumen of the channel being enclosed by the at least one inner side.
3. The respiratory therapy device according to at least one of the preceding items, wherein the at least one structural element is arranged in the lumen of the channel.
4. The respiratory therapy device of at least one of the preceding items, wherein the at least one structural element is arranged in and/or on at least one inner side of the housing.
5. The respiratory therapy device of to at least one of the preceding items, wherein the channel comprises an inlet channel, at least one flow channel, and at least one outlet channel, which have a communication connection to one another, the channel forming a flow path having a flow direction which extends from the inlet channel to the flow channel to the outlet channel.
6. The respiratory therapy device of at least one of the preceding items, wherein the structural element is formed as at least one partition wall and/or at least one counterbore.
7. The respiratory therapy device of any one of items 5 and 6, wherein the at least one partition wall is arranged at least in sections in a lumen of the flow channel and/or in a lumen of the outlet channel.
8. The respiratory therapy device of at least one of items 5-7, wherein the partition wall divides the flow channel and/or the outlet channel at least in sections at least partially into at least two channels.
9. The respiratory therapy device of at least one of items 5-8, wherein the partition wall is arranged in the channel such that a second tongue is formed in the flow channel or in the outlet channel.
10. The respiratory therapy device of at least one of items 5-9, wherein the at least one counterbore is arranged in an inner side of the housing in a region of the flow channel and/or the outlet channel.
11. The respiratory therapy device of at least one of items 5-10, wherein the counterbores are formed by a depression in at least one inner side of the housing.
12. The respiratory therapy device of at least one of items 5-11, wherein the counterbores have a depth of from about 0.5 mm to about 2.5 mm, preferably of from about 1 mm to about 2 mm, particularly preferably of about 1.4 mm.
13. The respiratory therapy device of at least one of items 5-12, wherein the counterbores are formed round and/or oval.
14. The respiratory therapy device of at least one of items 5-13, wherein the counterbores have a diameter of from about 1 mm to about 7 mm, preferably of from about 2 mm to about 5 mm, particularly preferably of about 4 mm.
15. The respiratory therapy device of at least one of items 5-14, wherein the inner sides have from 1 to about 100 counterbores, preferably from 1 to about 50, particularly preferably from about 4 to about 20 counterbores.
16. A fan unit for the respiratory therapy device according to at least one of the preceding items.

Although the present invention was described in detail on the basis of the exemplary embodiments, it is obvious for a person skilled in the art that the invention is not restricted to these exemplary embodiments. Rather, modifications are possible in such a way that individual features are omitted or other combinations of the described individual features can be implemented, if the scope of protection of the appended claims is not left. The present disclosure includes all combinations of the individual features presented.

List of reference signs

| | |
|---|---|
| 1 | fan unit |
| 5 | fan wheel |
| 6 | blade elements |
| 10 | housing |
| 11 | housing wall |
| 12 | channel |
| 13 | center axis |
| 16 | front part |
| 16a | outer side of the front part |
| 16i | inner side of the front part |
| 17 | rear part |
| 17a | outer side of the rear part |
| 17i | inner side of the rear part |
| 18 | connection joint |
| 20 | suction nozzle |
| 20a | suction nozzle outer side |
| 20i | suction nozzle inner side |
| 21 | suction nozzle wall |
| 22 | inlet opening |
| 24 | inlet channel |
| 25 | support ribs |
| 29 | suction nozzle flange |
| 30 | pressure nozzle |
| 30a | pressure nozzle outer side |
| 30i | pressure nozzle inner side |
| 31 | pressure nozzle wall |
| 32 | outlet opening |
| 33 | partition wall |
| 33B | width of the partition wall |
| 33H | height of the partition wall |
| 33L | length of the partition wall |
| 34 | outlet channel |
| 34H | height of the outlet channel |
| 35 | inner outlet channel |
| 35H | height of the inner outlet channel |
| 36 | outer outlet channel |
| 36H | height of the outer outlet channel |
| 37 | housing tongue |
| 38 | second tongue |
| 39 | pressure nozzle flange |
| 40 | opening |
| 41 | element |
| 42 | bore |
| 43 | receptacle unit |
| 44 | receptacle joint |
| 45 | end |
| 50 | flow channel |
| 52 | inner flow channel |
| 54 | outer flow channel |
| 60 | counterbore |
| 60D | diameter of the counterbore |
| 60T | depth of the counterbore |
| 70 | respiratory therapy device |
| 71 | operating unit |
| 72 | display unit |
| 73 | hose system |
| 74 | control unit |
| 75 | patient interface |
| 76 | interface |
| 80 | flow direction |

What is claimed is:

1. A respiratory therapy device, wherein the device comprises at least one fan unit for generating a respiratory airflow for carrying out respiratory therapy, the fan unit comprising a housing and at least one fan wheel rotatably mounted in the housing, and wherein respiratory air is transported through a channel formed inside the housing, the housing comprising at least one structural element which reduces sound emission.

2. The respiratory therapy device of claim 1, wherein the housing comprises at least one inner side and the channel comprises a lumen, the lumen of the channel being enclosed by the at least one inner side.

3. The respiratory therapy device of claim 2, wherein the at least one structural element is arranged in a lumen of the channel.

4. The respiratory therapy device of claim 2, wherein the at least one structural element is arranged in and/or on at least one inner side of the housing.

5. The respiratory therapy device of claim 1, wherein the channel comprises an inlet channel, at least one flow channel, and at least one outlet channel, which have a communication connection to one another, the channel forming a flow path having a flow direction which extends from the inlet channel to the flow channel to the outlet channel.

6. The respiratory therapy device of claim 1, wherein the structural element is formed as at least one partition wall and/or at least one counterbore.

7. The respiratory therapy device of claim 5, wherein the structural element is formed as at least one partition wall and/or at least one counterbore and the at least one partition wall is arranged at least in sections in a lumen of the flow channel and/or in a lumen of the outlet channel.

8. The respiratory therapy device of claim 5, wherein the structural element is formed as at least one partition wall and/or at least one counterbore and wherein the partition wall divides the flow channel and/or the outlet channel at least in sections at least partially into at least two channels.

9. The respiratory therapy device of claim 5, wherein the structural element is formed as at least one partition wall and/or at least one counterbore wherein the partition wall is arranged in the channel such that a second tongue is formed in the flow channel or in the outlet channel.

10. The respiratory therapy device of claim 5, wherein the structural element is formed as at least one partition wall and/or at least one counterbore and wherein the at least one counterbore is arranged in an inner side of the housing in a region of the flow channel and/or the outlet channel.

11. The respiratory therapy device of claim 5, wherein the at least one counterbore is formed by a depression in at least one inner side of the housing.

12. The respiratory therapy device of claim 5, wherein the at least one counterbore has a depth of from 0.5 mm to 2.5 mm.

13. The respiratory therapy device of claim 5, wherein the at least one counterbore has a depth of from 1 mm to 2 mm.

14. The respiratory therapy device of claim 5, wherein the at least one counterbore is formed round and/or oval.

15. The respiratory therapy device of claim 5, wherein the at least one counterbore has a diameter of from 1 mm to 7 mm.

16. The respiratory therapy device of claim 5, wherein the at least one counterbore has a diameter of from 2 mm to 5 mm.

17. The respiratory therapy device of claim 2, wherein the inner sides of the housing comprise from 1 to 100 counterbores.

18. The respiratory therapy device of claim 2, wherein the inner sides of the housing comprise from 1 to 50 counterbores.

19. A fan unit for the respiratory therapy device claim 1.
